(19) United States
(12) Patent Application Publication
(10) Pub. No.: US 2023/0113221 A1
(43) Pub. Date: Apr. 13, 2023

(54) METHOD FOR PRODUCING BANKABLE AND SUBCULTURABLE MATURE MICROGLIA

(71) Applicants: SUNGKWANG MEDICAL FOUNDATION, Seoul (KR); CHA UNIVERSITY INDUSTRY-ACADEMIC COOPERATION FOUNDATION, Pocheon-si (KR)

(72) Inventors: Min Soo KWON, Seoul (KR); Jong Seop RIM, Seongnam-si (KR); Tae Ho KONG, Seongnam-si (KR); Min Jung YOU, Anyang-si (KR); Ji Hye WON, Hwaseong-si (KR)

(73) Assignees: SUNGKWANG MEDICAL FOUNDATION, Seoul (KR); CHA UNIVERSITY INDUSTRY-ACADEMIC COOPERATION FOUNDATION, Pocheon-si (KR)

(21) Appl. No.: 17/906,165

(22) PCT Filed: Nov. 10, 2020
(86) PCT No.: PCT/KR2020/015709

§ 371 Date: Sep. 12, 2022

(30) Foreign Application Priority Data
Mar. 13, 2020 (KR) 10-2020-0031304

(51) Int. Cl. C12N 5/079

(52) U.S. Cl. CPC C12N 5/0622; C12N 2502/08; C12N 2500/30; C12N 2500/02

(57) ABSTRACT

The disclosure relates to a method of producing bankable and subculturable mature microglia, and according to a method according to an aspect, subculture and banking are possible, and freeze storage and thawing are also possible, and thus, it is possible to simply isolate and use only mature microglia whenever necessary. In addition, it is possible to dramatically reduce the number of subjects required for an experiment, and therefore, the method may contribute economically to all research or industrial fields related to microglia.

### TECHNICAL FIELD

The present disclosure relates to a method of producing bankable and subculturable mature microglia.

### BACKGROUND ART

Microglia are a type of glia located throughout the brain and spinal cord Microglia are derived from the mesoderm, activated by injury or invasion of foreign substances such as viruses, and are known to have various physiological functions such as antitumor activity, in addition to phagocytosis and tissue repair. In addition, microglia remove by phagocytosis waste such as amyloid β or dead cells caused by apoptosis or external damage.

In relation to diseases, microglia are known to be involved in chronic neurodegenerative diseases, such as Alzheimer's disease, Parkinson's disease, and Lou Gehrig's disease, and cerebral infarction associated with acute and subacute brain injury, and reactivity of microglia is known to be changed by toxic products accumulating in the brain represented by amyloid β.

Thus, it is necessary to use microglia or mature microglia for the development and evaluation of therapeutic agents for diseases related to microglia, and various methods are being tried for this purpose.

A method in the art of obtaining microglia includes i) culturing a microglial cell line. In this case, subculture and storage are possible, but the cell line is an immortalized cell line transformed with a virus, and there is a disadvantage in that characteristics of mature microglia are not reflected. ii) Secondly, there is a method using primary cultured microglia. Primary cultured microglia show more characteristics of mature microglia compared to the above-described cell line, however, primary culture microglia are impossible to subculture, difficult to store, and do not completely possess the genetic characteristics of mature microglia. Furthermore, in order to secure the number of cells for an experiment, there is a disadvantage of having to dissect neonatal subjects for every experiment. iii) Thirdly, there is a method of isolating microglia directly from the brain of a subject. In this case, mature microglia may be obtained, but characteristics of the cells are changed after isolation, and thus, it is difficult to culture the cells for a long time after isolation while maintaining the characteristics of the cells, subculture and storage are impossible, and there is also the disadvantage that a large number of experimental subjects are needed.

Therefore, there is an urgent need for a method capable of producing mature microglia which may be subcultured and stored.

### DESCRIPTION OF EMBODIMENTS

### Technical Problem

An aspect is to provide a method of producing mature microglia including: culturing a mixture including neuroepithelial cells and microglial precursors; and isolating microglia from the mixture.

Another aspect is to provide mature microglia produced according to the production method.

### Solution to Problem

An aspect is related to a method of producing mature microglia including: culturing a mixture including neuroepithelial cells and microglial precursors; and isolating microglia from the mixture.

The “neuroepithelial cell” refers to an epithelial cell differentiated to fit for a sensory organ, and refers to the part of the ectoderm that will become the nervous system. Neuroepithelial cells are formed during the development of a neural tube or a neural plate during the fetal brain development, and are primitive neural stem cells that have multipotency of being capable of differentiating into neurons and glial cells.

The “microglia” are cells involved in immunity in the brain which help survival of nerve cells by secreting nutrients, or protect the nerve cells from invasion of foreign substances by secreting substances that induce inflammation.

The mixture of neuroepithelial cells (NEC) and microglial precursors is not particularly limited within the range that the mixture includes neuroepithelial cells and microglial precursors, specifically, the mixture may be one isolated or peeled off from a subject's neuroepithelial layer or cerebral cortex, or more specifically, the mixture may be isolated or peeled off from a subject's neuroepithelial layer.

According to an aspect, isolating the mixture from a subject's neuroepithelial layer may be further included.

The “subject” refers to all living organisms, such as a rat, a mouse, a livestock, etc., except for humans, which may have neuroepithelial cells and microglial precursors. As a specific example, the subject may be a mammal including a rat, a mouse, etc., and more specifically, may be a rat, a mouse, etc.

Also, according to an aspect, the subject may be a fetus isolated from the uterus of a pregnant parent.

The fetus is not limited within the range that the parent is pregnant with the fetus, however, specifically, when the subject is a mouse, the subject may be a fetus 11.5 to 15.5 days old after the parents' sexual intercourse, when the subject is another animal, particularly a mammal, the fetus may be a fetus corresponding to the 11.5 to 15.5 days old fetus after the parents' sexual intercourse when the subject is a mouse. In addition, more specifically, the fetus may be a fetus about 12.5 days to about 14.5 days old after the parents' sexual intercourse, when the subject is a mouse; and when the subject is another animal, particularly a mammal, the fetus may be a fetus corresponding to the fetus about 12.5 days to about 14.5 days old after the parents' sexual intercourse when the subject is a mouse. In this regard, “correspondence” includes those having the same number of days, and includes those in the same stage of development based on the adult according to the gestation period or the fetal development of each subject.

The isolation from the uterus of the pregnant parent of the subject may be performed according to a known isolation method.

The neuroepithelial layer is not particularly limited within the range that it is a neuroepithelial layer in the head, brain, retina, etc., but for example, may be a neuroepithelial layer in the head or a side of the brain.

According to an aspect, the culturing may be culturing in the art or subculturing.

The culturing may differentiate or change the microglial precursors contained in the mixture into mature microglia as the period or number of passages increases. Specifically, when the culturing is subculturing, when microglia are isolated from the mixture after 2 to 6 passages, more specifically, 3 to 5 passages, the isolated microglia may be mature microglia.

In an aspect, the method of preparing mature microglia may further include: freezing and storing the mixture prior to isolating the microglia;

and thawing the mixture.

Proliferation and subculture are possible even when the mixture is stored frozen, and the microglia in the mixture maintain phagocytic and migratory ability, and maturity may also be maintained. Therefore, according to the method according to an aspect, it is possible to isolate and use only mature microglia anytime, anywhere, so that the number of subjects required for the experiment may be drastically reduced.

In an aspect, the isolation may be performed by using at least one method selected from the group consisting of a magnetic-activated cell sorting (MACS) system, a fluorescent-activated cell sorting (FACS) system and a shaking method, and specifically, may be performed by using a MACS system.

In an aspect, the microglia may express CD11 b, a marker of microglia.

In addition, the microglia may be one expressing at least one gene selected from the group consisting of IBA-1, PU1, TMEM119, P2RY12, CSF1R, MAFB, TREM2, Olfml3, Hexb, TGFbeta, MERTK, C1 QA, GPR34, and TGFBR1, markers of mature microglia, and specifically, the microglia may be one expressing at least one gene selected from the group consisting of IBA-1, and TMEM119.

In an aspect, the microglia may be capable of phagocytosis. Phagocytosis, a function of microglia, may appear equivalently or similarly to that of mature microglia.

In an aspect, the microglia may be in a ramified form. The ramified form is a cellular form of mature microglia, which may be observed through a microscope.

Another aspect provides mature microglia prepared according to the method for producing mature microglia.

The preparation method is as described before, and the mature microglia may express at least one gene selected from the group consisting of CD11b, IBA-1, Nestin, PU1, TMEM119, P2RY12, CSF1R, MAFB, TREM2, Olfml3, Hexb, TGFbeta, MERTK, C1 QA, GPR34, and TGFBR1.

In addition, the mature microglia may be capable of phagocytosis, and may be in a ramified form.

### Advantageous Effects of Disclosure

According to the production method according to an aspect, subculture and banking are possible, and freeze storage and thawing are also possible, and thus, it is possible to simply isolate and use only mature microglia whenever necessary. In addition, it is possible to dramatically reduce the number of subjects required for an experiment, and thus, the method may economically contribute to all research or industrial fields related to microglia.

### BRIEF DESCRIPTION OF DRAWINGS

FIG. 1A to 1D are diagrams illustrating isolation of neuroepithelial cells from the neuroepithelial layer and culturing the same, and confirming the presence of microglia among the mixed cells isolated from the neuroepithelial layer.

FIGS. 2A and 2B are diagrams comparing the neuroepithelial layer and the cerebral cortex as sources of microglia and illustrating isolation of microglia by using a magnetic-activated cell sorting (MACS) system.

FIG. 3 is a diagram confirming expression of major markers of purely isolated microglia through immunofluorescence staining.

FIGS. 4A and 4B are diagrams illustrating subculture of neuroepithelial cells including microglia and confirming phagocytic function of purely isolated microglia.

FIG. 5 is a diagram confirming unique characteristics of purely isolated microglia through immunofluorescence staining.

FIG. 6 is a diagram confirming unique characteristics of purely isolated microglia through qPCR.

FIG. 7A is a change of TMEM119 expression according to NEC passage and 7B is graphs comparing expression levels of major markers of fetal microglia, BV2 (microglia cell line), adult microglia, and purely isolated microglia after being subcultured.

FIG. 8A to 8C are diagrams confirming changes according to culture period of purely isolated microglia.

FIG. 9 is a diagram confirming the highest number of microglia in neuroepithelial cells isolated at day 13.5, through immunochemical staining.

FIG. 10 is a diagram confirming that microglia proliferate and mature when a 13.5-day-old neuroepithelial cell layer is isolated and then cultured for 21 days.

FIG. 11 is a diagram confirming through flow cytometry that the percentage of microglia (CD11 b) is increased when the fetal neuroepithelial cell layer is isolated and then cultured for 21 days.

FIG. 12 is a diagram confirming subculturability after isolating the neuroepithelial layer of mouse E13.5 and culturing the same for 21 days.

FIG. 13 is a diagram confirming that microglia have proliferative ability when neuroepithelial cells are cultured for 21 days and then subcultured.

FIG. 14 is a diagram confirming that microglia (NEC-MG) express microglial markers (IBA-1, CX3CR1, TMEM119), wherein the microglia are obtained by isolating only microglia using a MACS system after culturing an neuroepithelial cell layer for 21 days.

FIG. 15 is a diagram confirming maturity of microglia isolated from a neuroepithelial layer.

FIG. 16 is a diagram confirming that maturity of microglia is maintained even when subcultured and continuously cultured.

FIG. 17 is a diagram confirming microglial markers and phagocytic ability following subculture and continuous culture.

FIG. 18A to 18C are diagrams confirming the phagocytic ability of microglia.

FIG. 19 is a diagram confirming the migratory ability of microglia.

FIG. 20 is a diagram confirming the ability of microglia to secrete cytokines when treated with lipopolysaccharide (LPS).

FIG. 21 is a diagram confirming the ability to secrete inflammatory and anti-inflammatory cytokines after LPS treatment.

FIGS. 22A and 22B are diagrams confirming microglia isolated from a neuroepithelial layer stored frozen and thawed.

FIG. 23 is a diagram confirming the maturity level of microglia isolated from a neuroepithelial layer that has been stored frozen and thawed.

### MODE OF DISCLOSURE

Hereinafter, the present disclosure will be described in more detail through examples. However, these examples are intended to illustrate the present disclosure, and the scope of the present disclosure is not limited to these examples.

### Example 1

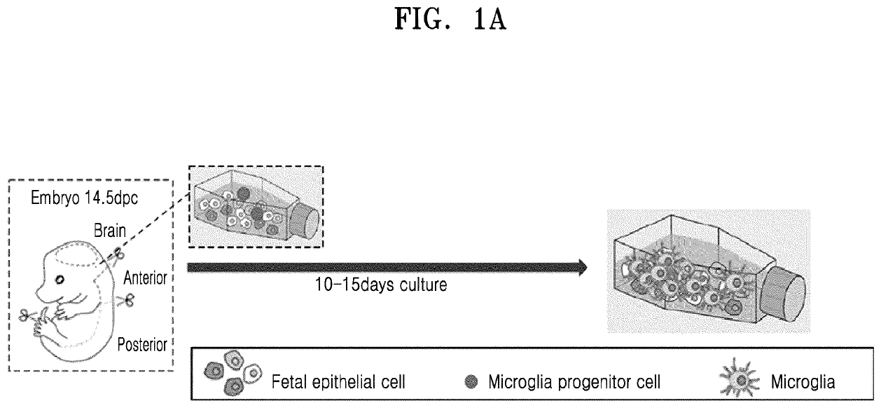

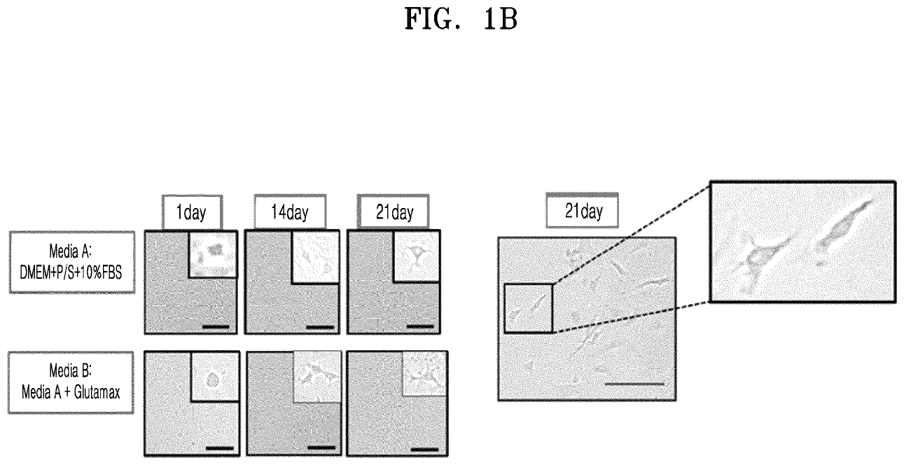

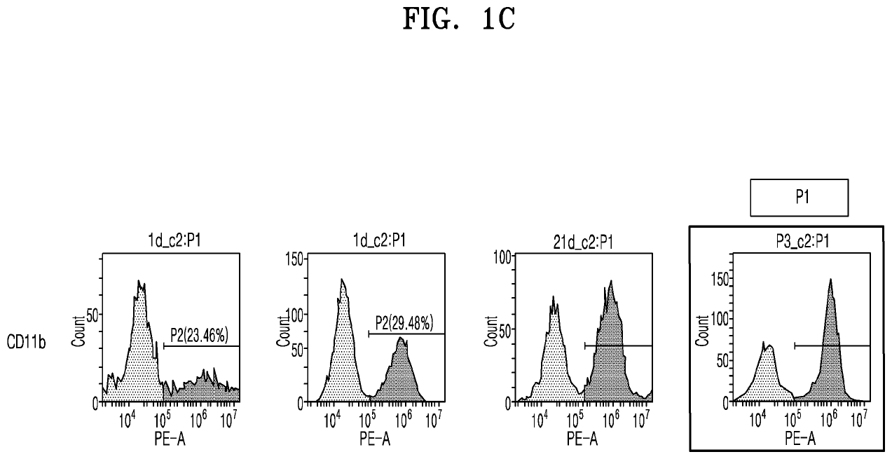

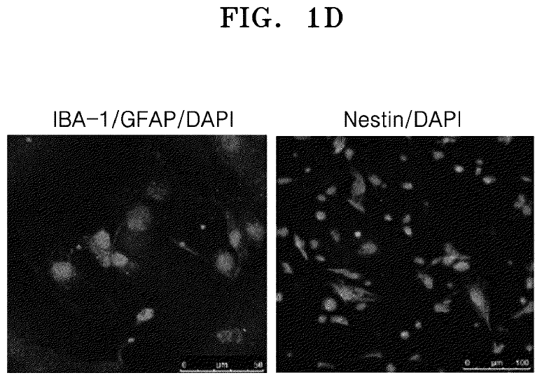

1. Isolation of Neuroepithelial Cells (NEC), Culture and Confirmation of Presence of Microglia (FIG. 1A)

In order to obtain neuroepithelial cells, 13.5-day-old embryos were isolated from the uterus, and the neuroepithelial layer present on the head was dissected, and single cell suspension was performed in Hanks' Balanced Salt Solution (HBBS; gibco, cat. #14170-112).

Thereafter, the cells contained in HBSS were centrifuged at 1,200 rpm for 3 minutes, the HBBS medium was removed, and 1 ml of 1×Trypsin-EDTA (gibco, cat. #15400-054) was added.

After incubating for 3 minutes, the sample was centrifuged at 1,200 rpm for 5 minutes to sink the cells, and then the medium was refreshed with a new medium (DMEM (gibco, cat. #11995-065), 10% heat-inactivated fetal bovine serum (gibco, cat) #16000-044), 0.1× GlutaMAX™ (gibco, cat. #35050-061) and 1% penicillin/streptomycin (Gibco, cat #15140122)).

Three sets of samples were cultured in a 75T-flask coated with Poly-D-lysine hydrobromide (SIGMA, cat #P7280) in a 5% CO₂ incubator at 37° C. (one set means 10 to 12 pups, and a 25T-flask is used when only one set is cultured).

One day later, the culture medium was refreshed, and when the flask reached a state of cell saturation after culturing for 15 days or more, the morphological change (microglial induction process) of the cultured cells was confirmed by using a phase-contrast microscope (Nikon). Images were taken with a DS-Ri2 digital camera (Nikon Instech). As a result, it was possible to observe ramified cells through a microscope (FIG. 1B).

In addition, as a result of culturing the cells in a medium containing and not containing GlutaMAX, a glutamine supplement, it was confirmed that microglia were more rapidly grown when the cells were cultured in a medium containing GlutaMAX (FIG. 1B).

In addition, since CD11 b is a microglial marker expressed only in microglia among neuroepithelial cells, flow cytometry analysis was performed using anti-CD11 b antibodies (BD, cat. #553311).

Specifically, monoclonal antibodies conjugated with a fluorescent dye specific to mouse CD11 b (cat. #553311; BD Biosciences, San Jose, Calif.) were used. The cells were washed with phosphate-buffered saline (PBS; cat. #14190-144, gibco) free of calcium-magnesium, and cultured for 5 minutes in cell staining buffer (cat. #420201; BioLegend) at 4° C. The antibodies were incubated with cell suspension at 4° C. for 30 minutes, washed with PBS. The expression rate of CD11 b was calculated with the fluorescence intensity of each fluorochrome. All data were collected in a CytoFLEX flow cytometer (Beckman) and analyzed with a CyExpert software (Beckman).

As a result of the flow cytometry, the number of CD11 b-positive cells was confirmed to be increased to 55.64%, and was confirmed to be 59.55% when measured again after being subcultured (FIG. 1C).

In addition, to confirm whether the CD11b-positive cells are microglia, microglial markers were identified through immunofluorescence staining.

Specifically, primary cultured microglia were seeded on glass coverslips in a 24-well plate. Cells were washed with PBS, fixed with 4% formaldehyde, and permeabilized with 0.5% of Triton X-100 for 5 minutes. ProLong™ Gold Antifade Mountant with DAPI (Invitrogen, cat. #P36931) reagent was used to fix the cells, store the cells for a long-term, and to maintain fluorescent staining, and indirect immunofluorescence was performed using the following antibodies as primary antibodies: rabbit anti-GFAP antibody (abcam, cat. #ab7260), goat anti-IBA-1 antibody (abcam, cat. #ab48004), rabbit anti-IBA-1 antibody (wakko, cat. #019-19741) and rabbit anti-Nestin antibody (abcam, cat. #ab6142). Cells were incubated overnight at 4° C. with primary antibodies diluted with 0.5% Triton X-100 in PBS containing 1% bovine serum albumin. After rinsing 3 times with PBS for 5 minutes, secondary antibodies (anti-rabbit IgG H&L (Alexa) Fluor, cat. #ab150073), anti-goat IgG H&L (Alexa Fluor, cat. #ab150129)) conjugated with Alexa 488 (invtrogen, cat. #A11034, #A21202) or Alexa-594 (invtrogen cat. #A21203) were used for detection. Nuclei were counterstained with 4′,6-diamidino-2-phenylindole (DAPI; Sigma, Mo., USA). Cells without the addition of primary antibodies were used as negative controls. Fluorescence images were taken with a confocal microscope (TCSSP5 II, Leica microsystems, Wetzlar, Germany).

As a result of immunofluorescence staining (with DAPI), the expression of IBA-1, a microglial marker, was confirmed, but the expression of GFAP, a marker of astrocytes, could not be confirmed, and Nestin, a marker of neuroepithelial cells, was confirmed to be expressed (FIG. 1D).

As a result, by culturing neuroepithelial cells, it was confirmed that microglia proliferated and matured over time.

2. Comparison of Cerebral Cortex and Neuroepithelial Layer as Sources of Microglia and Isolation of Pure Microglia.

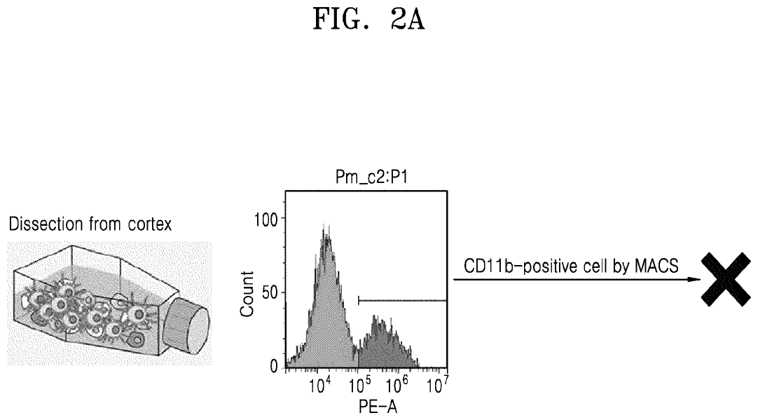

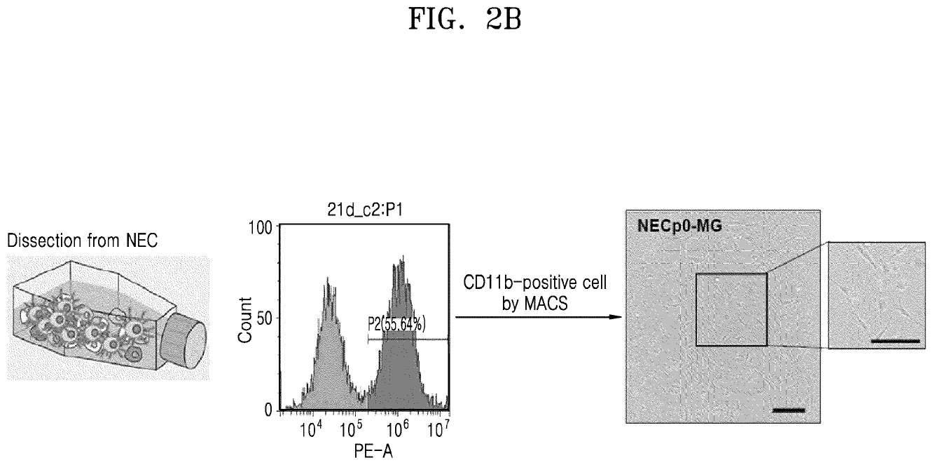

Microglia from cerebral cortices of 13.5-day-old mouse embryos were cultured in the same way as in Example 1, but CD11b-positive cells were relatively few at 23.38% (FIG. 2A).

In addition, the MACS system was used to isolate pure CD11 b-positive cells from NEC.

Specifically, the number of cells was first counted, and when the total number of cells was 1×10⁷, 10 μl of CD11 b and PBS and 90 μl of 0.5% BSA buffer were incubated at 4° C. for 15 minutes. About 1 ml to 2 ml of PBS and 0.5% BSA buffer were added, and centrifuged at 300 g for 10 minutes. The supernatant was removed, and when the number of cells was 1×10⁷, 500 μl of PBS and 0.5% BSA buffer were added.

After this, CD11b MACS beads (MS Columns (Miltenyi, cat. #130-042-201), CD11 b (Miltenyi, cat. #130-093-636)) were used for positive selection (130-093-636, Miltenyi) (isolation by using LS columns is also possible). As described for mouse experiments, RNA was immediately extracted and analyzed for purity.

As a result of observing the cells isolated by MACS under a microscope (bright field), it was found that only cells in a ramified form were purely isolated (FIG. 2B), and the number of cells was 5×10⁵ (Table 1).

In addition, in order to isolate pure CD11 b-positive cells from NEC, the shaking method may be used in addition to the MACS system, and the specific method is as follows.

The sample was incubated with shaking for 45 minutes at 50 rpm in a Shaking Incubator (JEIOTECH, KOREA). Then, 14 ml of the medium was removed and 14 ml of fresh medium was added so that the final volume was 21 ml, and the sample was incubated in a 5% CO₂ incubator at 37° C. for 3 hours. In addition, the sample was shaken at 160 rpm for 2 hours, put in a 50 ml conical tube, and centrifuged at 1,200 rpm for 8 minutes. After that, the number of pellets (microglia) was counted, and the shaking isolation method was also confirmed to be possible (Table 1).

TABLE 1

| MACS system | Cell number |
|---|---|
| Microglia isolated from subjects' | 5 × 10⁵ cells |
| brains (N = 2) | |
| Shaking method | Cell number |
| Primary cultured microglia (pup 15) | 5 × 10⁵ cells |
| NECp0-MG (pup15-16) | 8-9 × 10⁵cells |
| NECp2-MG | 2 × 10⁶ cells |

3. Confirmation of Expression of Key Markers in Purely Isolated Microglia

The expression patterns of microglial markers PU1, IBA-1, and TMEM119 according to culturing period of NEC were confirmed by performing immunofluorescence staining in the same manner as in Example 1 (Rabbit anti-TMEM119 antibody (abcam, cat. #ab209064) and mouse anti-Pu.1 antibody (abcam, cat. #ab88082)).

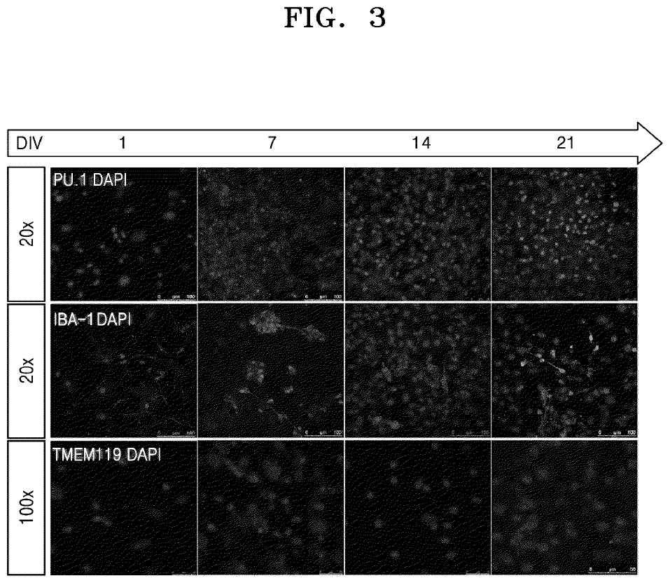

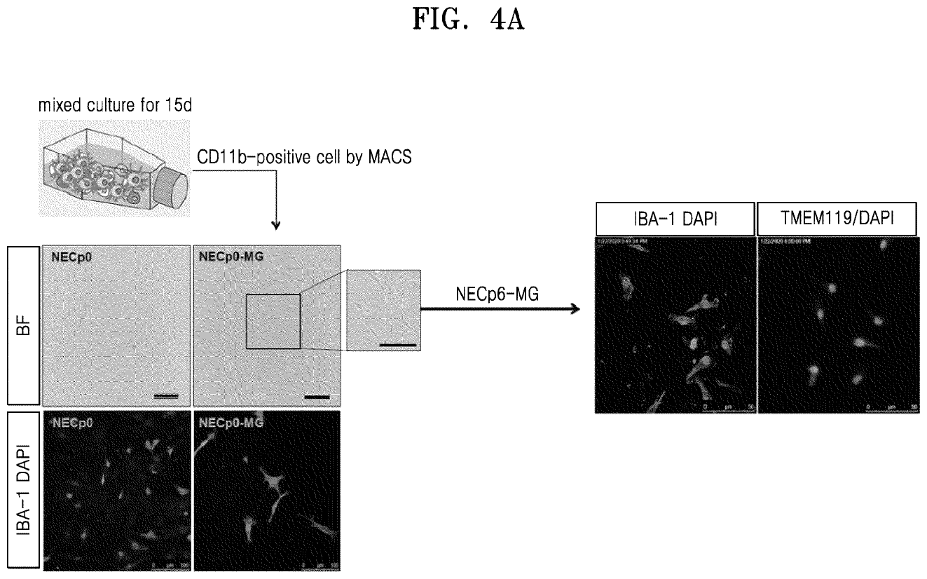

As a result, the expression levels of PU1, IBA-1, and TMEM119 all increased over time, and in particular, it was confirmed that the expression of TMEM119 (a marker of mature microglia), which is expressed only in adult mouse microglia, started to slowly increase. (FIG. 3).

4. Subculturing Neuroepithelial Cells Including Microglia and Confirmation of Phagocytic Function of Purely Isolated Microglia

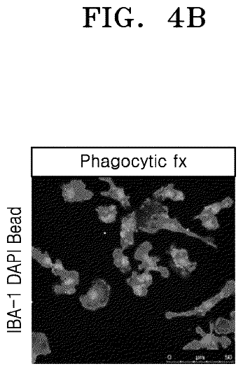

IBA-1 and TMEM119 markers were confirmed in CD11 b-positive cells (microglia) (NECp0), which are isolated by the MACS system from the NEC cultured for 15 days or more, and microglia (NECp6) isolated by the MACS system from the NEC passaged 6 times, and in particular, expression of TMEM119 in NECp6-MG was confirmed by DAPI staining. As a result, it was confirmed that the expression levels of IBA-1 and TMEM119 were increased in NECp6 than in NECp0 (FIG. 4A).

In addition, to confirm whether the function of microglia is well performed, phagocytosis, a major function of microglia, was confirmed in NECp0-MG.

Specifically, to investigate phagocytic activity of microglia, microglia at a concentration of 2×10⁵ cells/mL were seeded on 12-mm coverslips of a 24-well cell culture dish. The cells were cultured with 2 μl of red-stained Fluoresbrite microspheres with red fluorescent latex beads (2 μm, cat. #L3030-1 ML, Sigma-Aldrich, St. Louis, Mo., USA) at 37° C. for 2 hours. Then, 2 ml of cold PBS was added to stop the phagocytosis. The cells were washed twice with cold PBS and counterstained with DAPI. Cells were analyzed by using a confocal microscope (TPS SP5 II, Leica). The number of phagocytosed beads per cell indicates phagocytic activity.

As a result, the red beads (SIGMA, cat. #L3030-1 ML) were phatocytosed and the intracellular yellow beads were confirmed (FIG. 4B).

5. Confirmation of Unique Characteristics of Purely Isolated Microglia

In the microglia (NECp1-MG, NECp3-MG) prepared as described above, expression of IBA-1 and TMEM119, which are markers expressed in adult microglia, was confirmed by immunofluorescence staining in the same manner as in Example 1, and the expression were compared with that of adult microglia and fetal microglia.

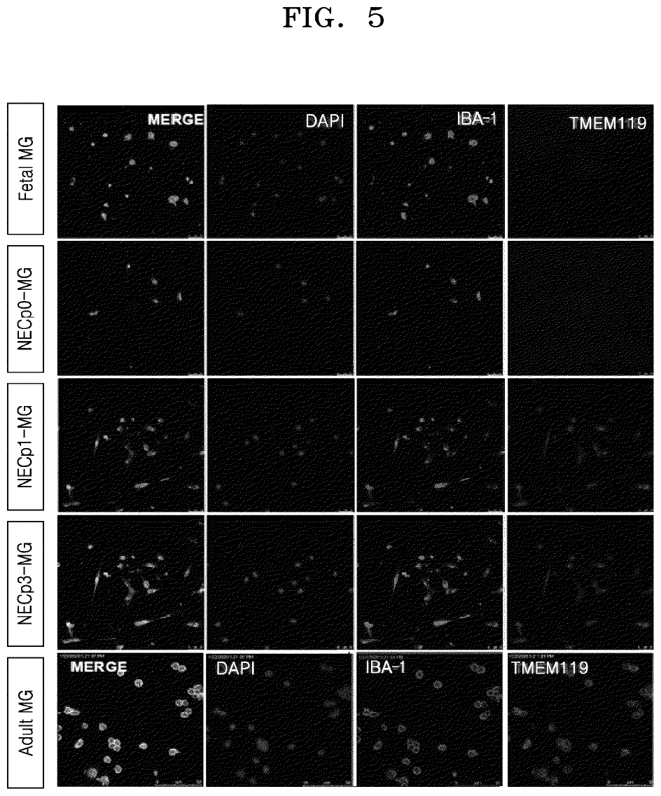

As a result, it was possible to confirm clearly more expression of IBA-1 and TMEM119 in NECp1-MG and NECp3-MG than in fetal microglia (FIG. 5).

In addition, expression analysis of microglial signature genes (TMEM119, P2RY12, CSF1R, MAFB, TREM2, Olfml3, Hexb, TGFbeta) was performed through quantitative reverse transcriptase polymerase chain reaction (qPCR).

Specifically, total RNA was extracted using TRIzol reagent (Invitrogen), and evaluated by using a DS-11 Series spectrophotometer/fluorometer (DeNovIx, DS-11 FX). cDNA was synthesized using the RevertAid First Strand cDNA synthesis kit (Thermo). To evaluate the characteristics of microglia of NECp-MG, expressions of TREM2 (Table 2 below), Olfml3 (Table 2 below), P2RY12 (Qiagen, Germany, PPM04913C), TMEM119 (PPM28876A), CSF1R (PPM03625F), MAFB (PPM05266A), HEXB (PPM27125A) and TGFB (PPM02991B) genes were analyzed. cDNA was amplified using a Power SYBR Green PCR Master Mix together with the primers in Table 1 below and the primers purchased from Qiagen, by using the Applied QuantStudio1 System (Thermo) for 10 minutes at 95° C. and performing 40 cycles of 15 seconds at 95° C. and 1 minute at 60° C. Melting curves were generated to test the specificity of the amplification. Relative quantity (RQ) levels were calculated by the 2-ΔΔ Ct method using GAPDH (Table 2 below) as an internal standard control group. Results were obtained by performing 3 independent experiments on independent cell batches.

TABLE 2

| | | | | Sequence |
|---|---|---|---|---|
| Mouse | Primer | | Sequence | No. |
| 1 | GAPDH | GAPDH-F | CATGGCCTTCCGTGTTCCTA | 1 |
| | | GAPDH-R | GCGGCACGTCAGATCCA | 2 |
| 2 | olfml3 | olfml3-F | CTGCTGCTCCTCTTCTTTTT | 3 |
| | | | G | |
| | | olfml3-R | CTACTCTGATCCTGGCATTG | 4 |
| 3 | trem2 | trem2-F | TGGGACCTCTCCACCAGTT | 5 |
| | | trem2-R | GTGGTGTTGAGGGCTTGG | 6 |

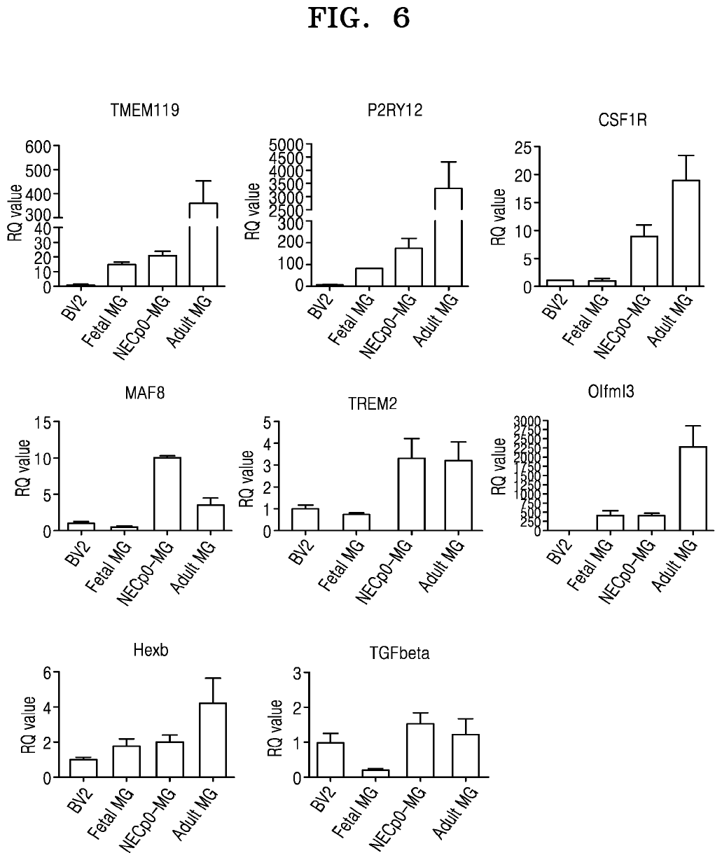

As a result of the qPCR, it was confirmed that genes were expressed in NECp0-MG at a level similar to that of adult microglia (FIG. 6).

6. Optimization of the Number of Passages to Obtain Mature Microglia

It was confirmed as shown in FIG. 5 that isolation of microglia after subculture of NEC increases the expression level of TMEM119. Thereafter, qPCR was performed as in Example 5 to confirm the optimized number of passages. Primers for additionally used markers (MERTK (Qiagen, Germany, PPM34425A), C1QA (PPM24525E), GPR34 (PPM04860A) and TGFBR1 (PPH00237C)) were purchased from Qiagen.

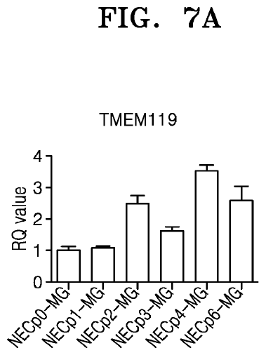

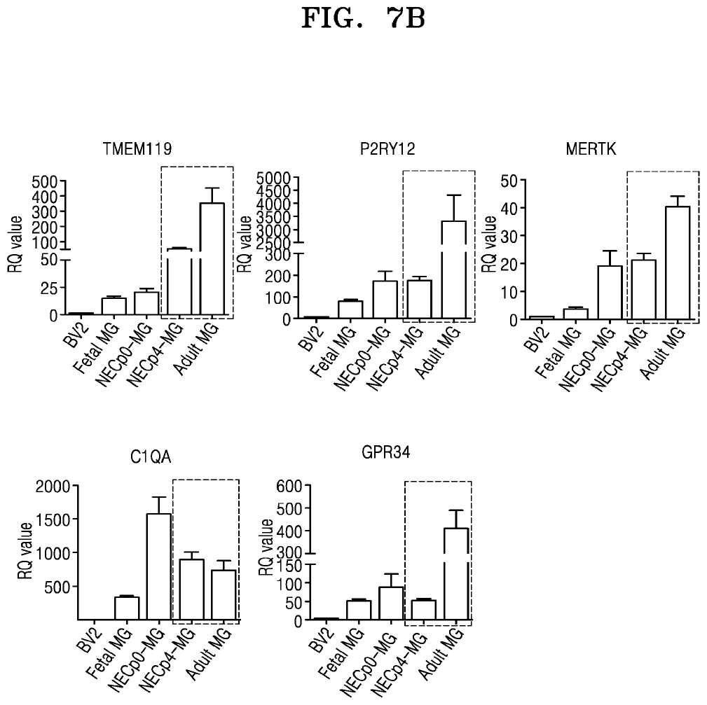

As a result, it was confirmed that the expression of TMEM119, a representative marker of adult microglia, was highest when the microglia were isolated at NEC passage 4 (NECp4-MG), and when isolated at passage 6 (NECp6-MG), and the expression level was confirmed to be partially decreased (FIG. 7A).

Expressions of microglial signature genes (TMEM119, MERTK, C1QA, GPR34, and TGFBR1) in NECp0-MG, NECp4-MG were compared with that in fetal microglia, BV2 (microglial cell line), and adult microglia, and it was found that the expression of the major genes (especially TMEM119) at least in NECp4-MG was similar to that of adult microglia (FIG. 7B).

7. Confirmation of Changes According to the Culture Period of Purely Isolated Microglia

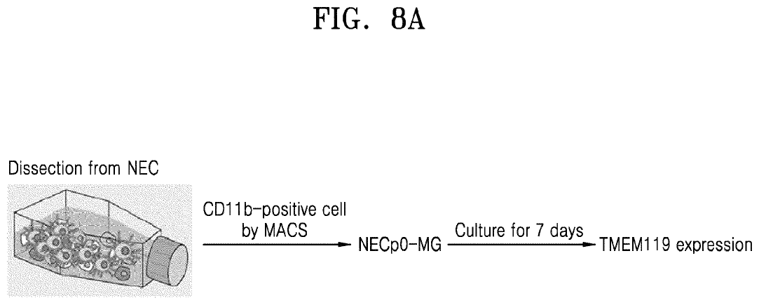

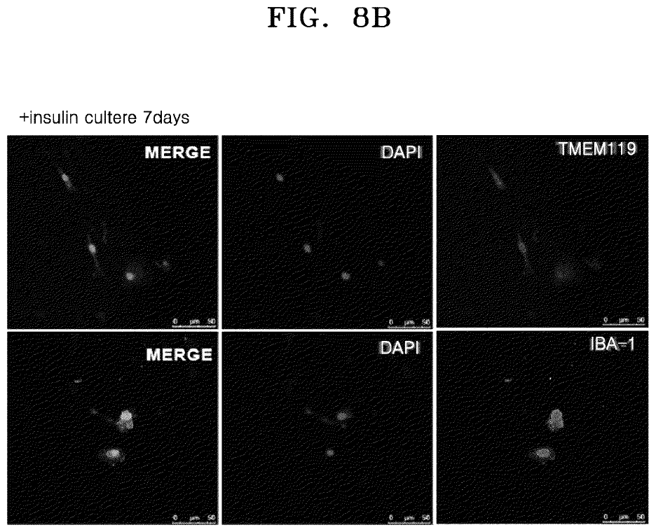

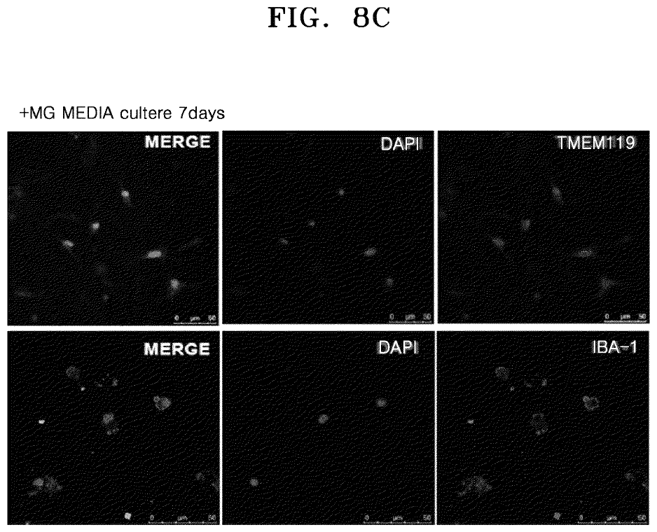

It was confirmed by immunofluorescence staining whether the microglia isolated from NEC maintained their characteristics even during the culture period (FIG. 8A). TMEM119 and IBA-1 were cultured for 7 days in insulin culture medium (DMEM (gibco, cat. #11995-065), 10% heat-inactivated fetal bovine serum (gibco, cat. #16000-044), 0.1× GlutaMAX™ (gibco, cat. #35050-061), 5 μg/ml Insulin solution human (sigma, cat. #19278-5ML) and 1% penicillin/streptomycin (Gibco, cat #15140122)) and microglia (MG) culture medium (DMEM (gibco, cat. #11995-065), 1% penicillin/streptomycin (gibco, cat #15140122), 2 ng/ml Human TGF-b2 (Peprotech, Cat. #100-35B), 100 ng/ml Murine IL-34 (R&D Systems, Cat. #5195-ML/CF) and 1.5 μg/ml Ovine wool cholesterol (Avanti Polar Lipids, Cat. #700000P)).

Thereafter, the expression of IBA-1 and TMEM119 was confirmed through immunofluorescence staining as in Example 1, and it was confirmed that the expression of IBA-1 and TMEM119 was well maintained despite continuous culture for 7 days (FIGS. 8B and 8C).

### Example 2

The materials and methods of the experiments in Example 2 are the same as in Example 1 above.

*1311. Comparison of number of microglia in neuroepithelial cells (NEC) according to age of mice

Neuroepithelial cells were isolated in the same manner as in Example 1, but were separated from 10.5-day-old mouse embryos, 13.5-day-old mouse embryos, and 17.5-day-old mouse embryos.

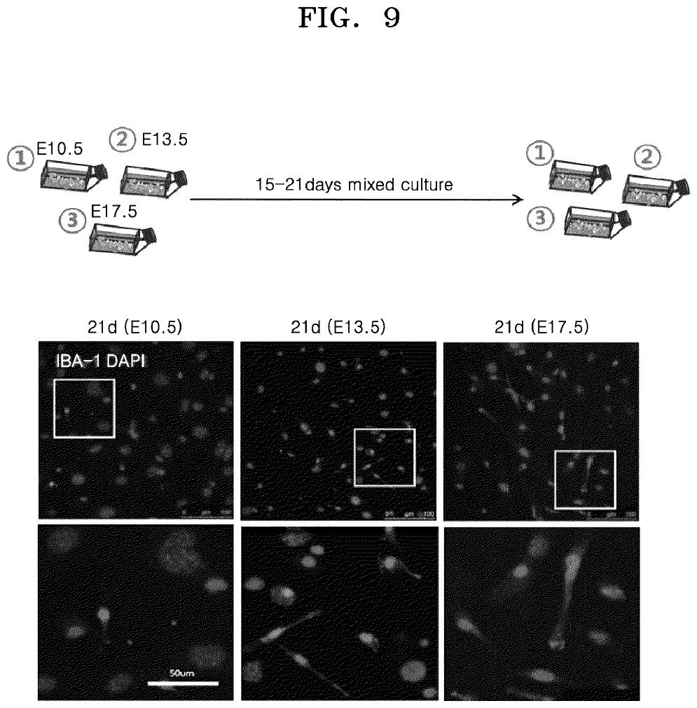

As a result, it was confirmed by immunochemical staining that the number of microglia was the highest in neuroepithelial cells isolated at 13.5 days of the embryonic stage (FIG. 9).

When compared with the existing microglia in vitro model, not only subculture was possible, but the culturing also showed a yield 10-fold or higher (Table 3 below).

TABLE 3

| | | | | CD11b |
|---|---|---|---|---|
| Cell | | DIV | | positive cell |
| Fetal microglia | — | 10-12 day | Postnatal | <5 × 10⁵ cells |
| | | | (p0-3), N = 10 | |
| Fetal microglia | | 12-14 day | Postnatal | <4 × 10⁵ cells |
| | | | (p0-3), N = 8 | |
| NEC-MG | | 12-14 day | E13, 5, N = 10 | <2 × 10⁶ cells |
| NEC-MG | P2 | 28 day | | <5 × 10⁶ cells |
| Adult microglia | — | — | 8-9 w, N = 1 | 5 × 10⁵ cells |
| Adult microglia | — | — | 10-11 w, N = 1 | 6 × 10⁵ cells |

2. Changes According to 21 Days of Culture of Isolated Neuroepithelial Cell Layer

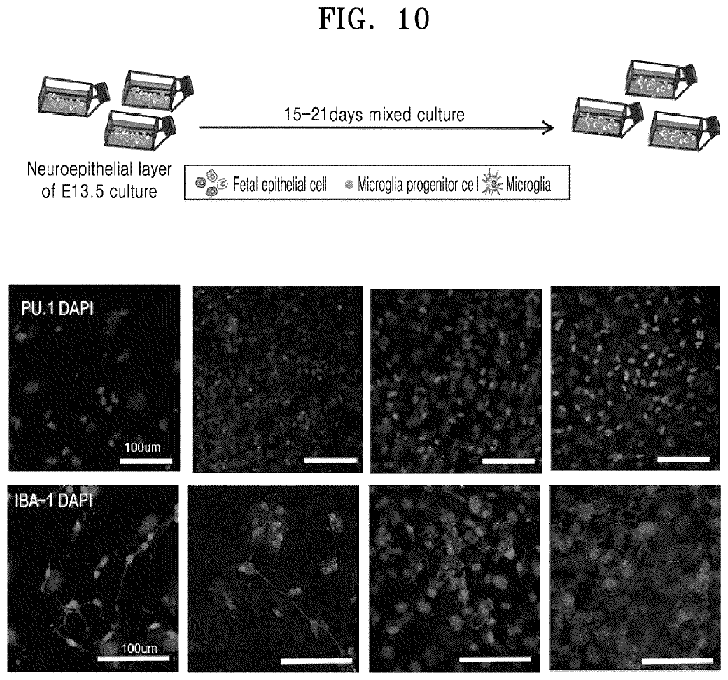

When a 13.5-day-old neuroepithelial cell layer was isolated and cultured for 21 days, it was confirmed that microglia proliferated (PU1 positive cells increased, IBA-1 positive cells in a ramified form increased) and matured (TMEM119 expression) (FIG. 10).

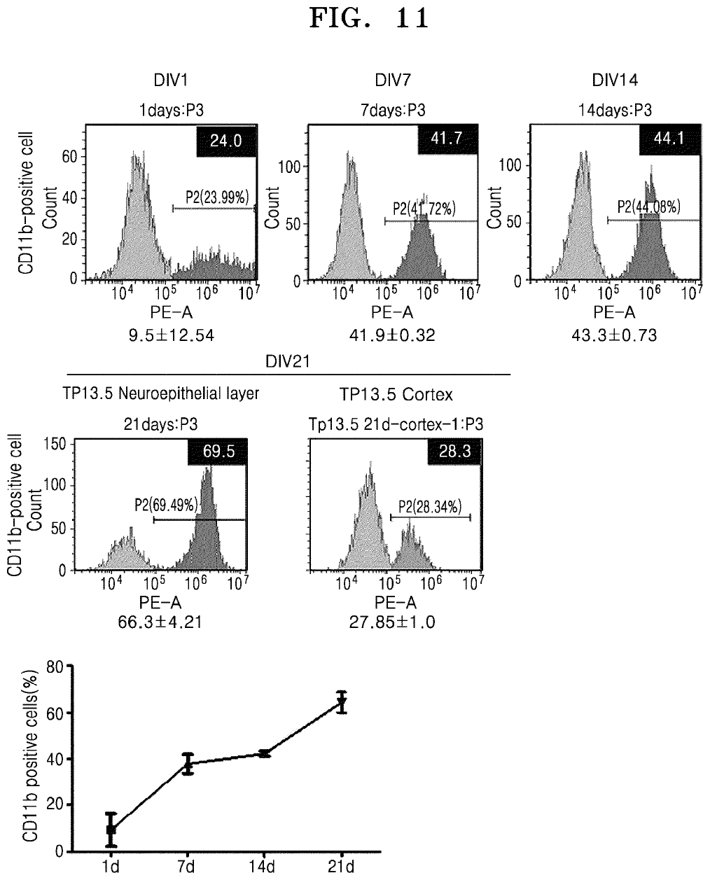

In addition, it was confirmed through flow cytometry that the percentage of microglia (CD11 b) was increased when the fetal neuroepithelial cell layer was isolated and cultured for 21 days. Specifically, it was possible to obtain about 70% of microglia (CD11 b positive cells) out of the entire cells when cultured for 21 days, however, when microglia were isolated from the cerebral cortex instead of the neuroepithelial layer at the same time (E13.5), only about 28.3% of microglia were obtained even after 21 days of culture (FIG. 11).

3. Confirmation of Subculturability of Isolated Neuroepithelial Layer

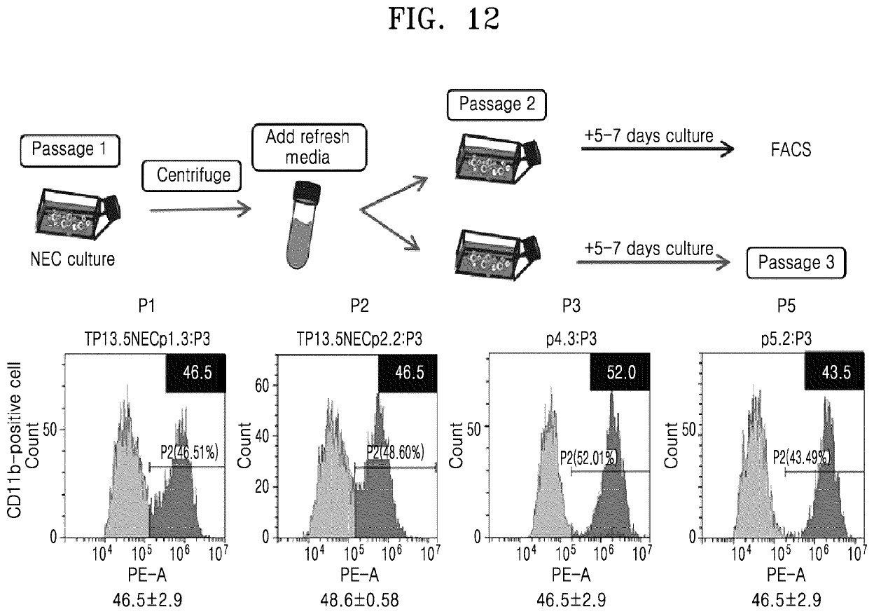

After neuroepithelial layers from 13.5 days old mice were isolated and subcultured for 21 days, it was confirmed that subculture was possible, and about 50% or more of microglia were obtained during the subculture (FIG. 12).

4. Confirmation of Proliferative Ability of Microglia after Subculture of Neuroepithelial Cells

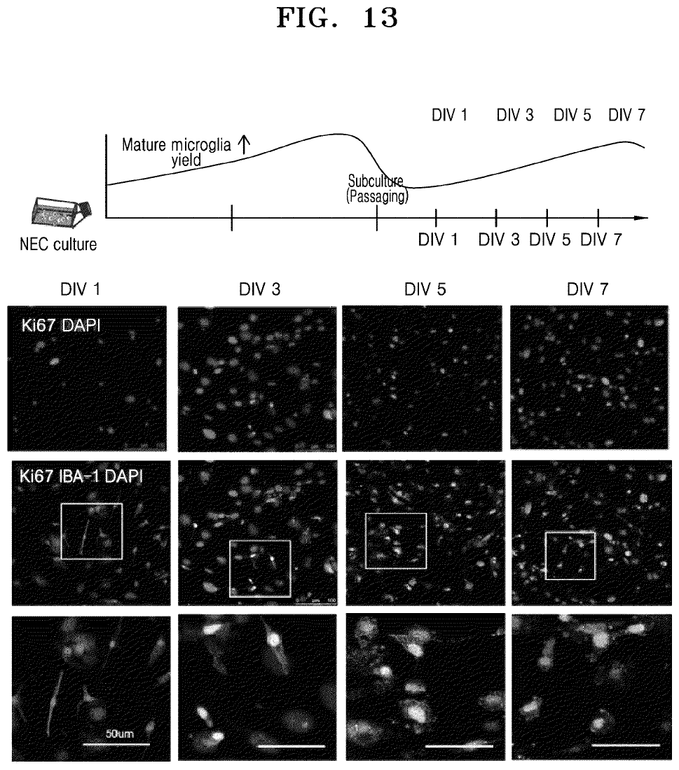

When neuroepithelial cells were cultured for 21 days and then subcultured, it was confirmed that the microglia had a proliferative ability (number of Ki67 positive cells increased, and Ki67 and IBA-1 double positive cells increased, FIG. 13).

In addition, it was shown that the percentage of microglia present was kept constant even after the subculture, which means that the method according to an aspect is culturing technology of microglia, in which subculturing is possible.

5. Confirmation of Isolated Microglia and Confirmation of Maturity

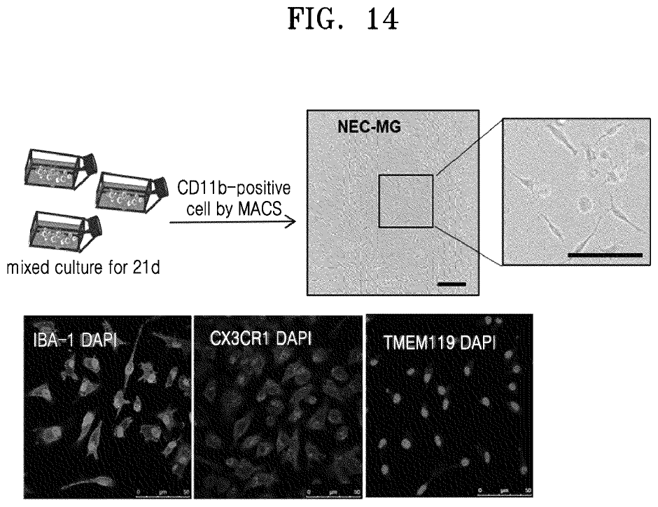

It was confirmed that microglia (NEC-MG) express microglial markers (IBA-1, CX3CR1, TMEM119), wherein the microglia were obtained by isolating only microglia using the MACS system after culturing the neuroepithelial cell layer for 21 days (FIG. 14).

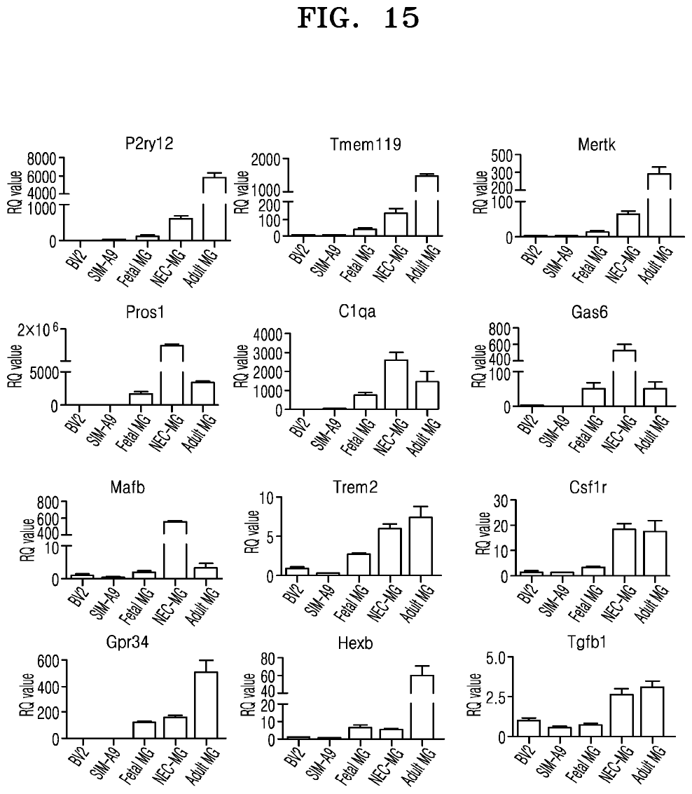

In addition, it was confirmed that NEC-MG showed a higher level of expression of genes of mature microglia compared to microglial cell lines (BV2, SIM-A9), microglia models in the art, and primary cultured microglia (immature microglia fetal microglia); and Pros1, C1qa, Gas6, Trem2, Csf1r, and Tgfb1 were expressed at a level similar to that of adult microglia (FIG. 15).

6. Confirmation of Maintenance of Maturity and Phagocytic Ability of Microglia

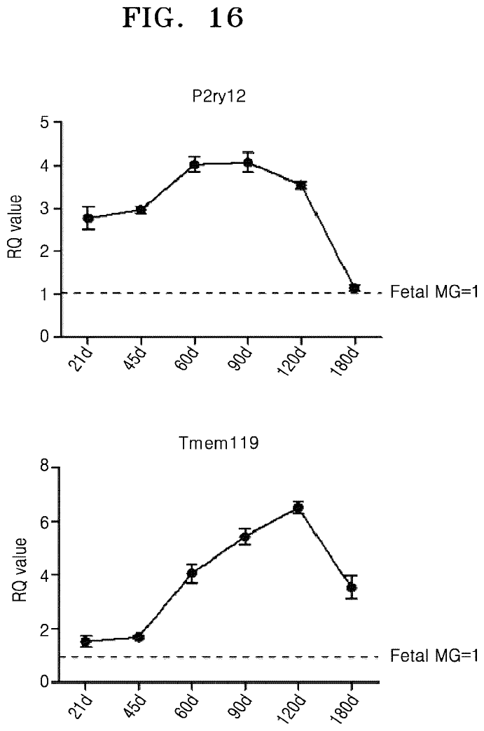

It was confirmed that expression of the representative genes of mature microglia (P2ry12, Tmem119) was maintained up to at least 180 days even when the neuroepithelial layer was subcultured and continuously cultured (FIG. 16).

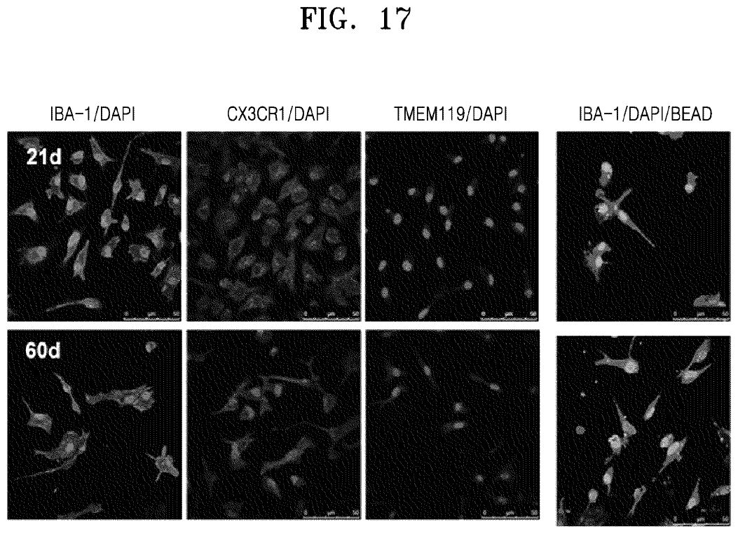

In addition, it was confirmed that not only microglial markers (IBA-1, CX3CR1) were expressed in microglia isolated after subculture, but also TMEM119, expressed in mature microglia, showed a higher expression pattern as passaged, and the microglia were also confirmed to have phagocytic activity (FIG. 17).

7. Confirmation of Phagocytic and Migratory Ability of Microglia Isolated from Neuroepithelial Layer

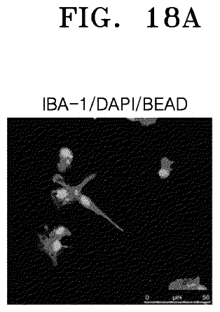

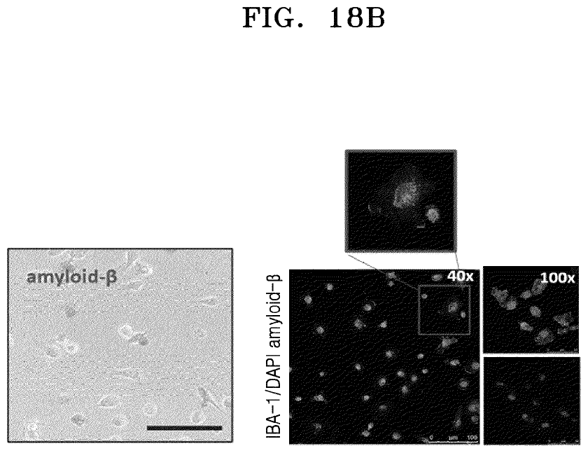

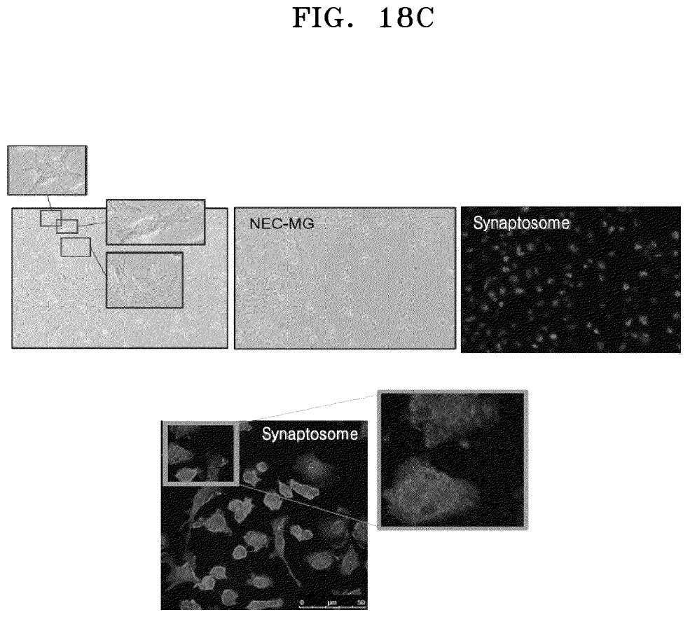

Fluorescent beads (FIG. 18A), amyloid-beta (FIG. 18B), and synaptosome (FIG. 18C) were treated to confirm the phagocytic ability of NEC-MG, and phagocytic activity was confirmed (FIG. 18).

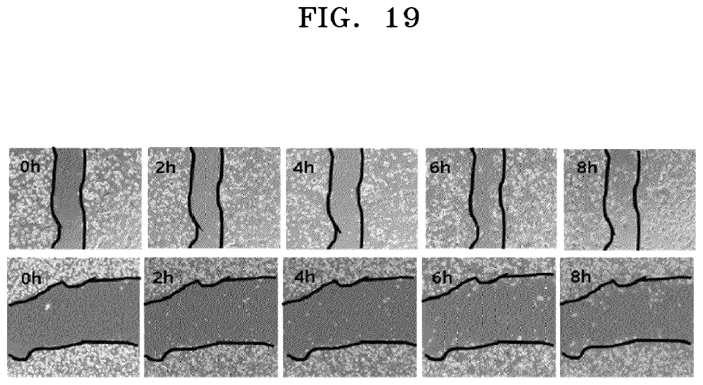

In addition, migratory ability of NEC-MG was also confirmed through wound healing assay (FIG. 19).

8. Confirmation of Cytokine-Secretion Ability of Microglia

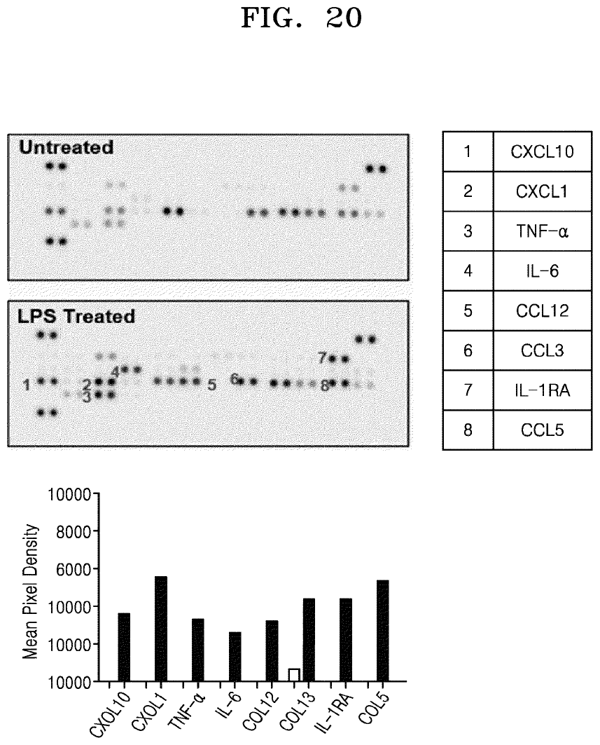

It was confirmed that NEC-MG is able to secrete various cytokines when 100 ng/ml of LPS was treated for 24 hours (FIG. 20).

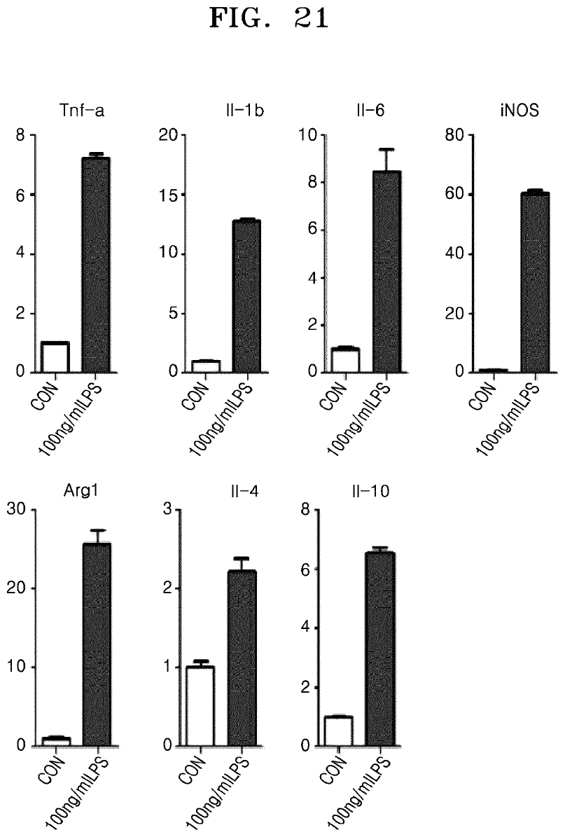

In addition, it was confirmed through qPCR that NEC-MG after LPS stimulation increased mRNA levels of inflammatory and anti-inflammatory cytokines (FIG. 21).

9. Confirmation of Proliferation and Subculturability when Neuroepithelial Layer is Stored Frozen

The purpose of the experiment was to determine whether proliferation and subculture were possible even when the neuroepithelial layer isolated from a 13.5-day-old embryo was stored frozen and then thawed and cultured.

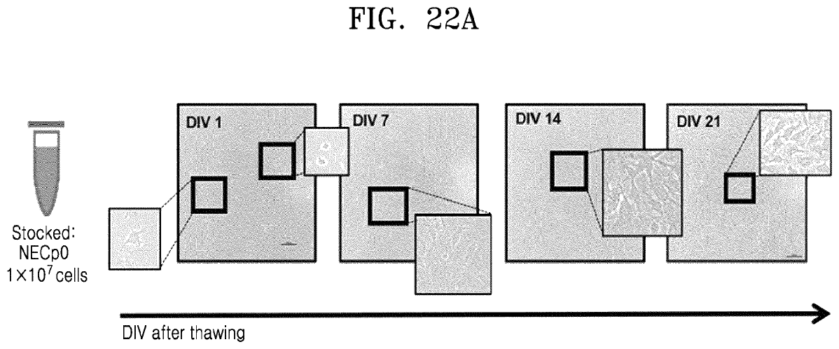

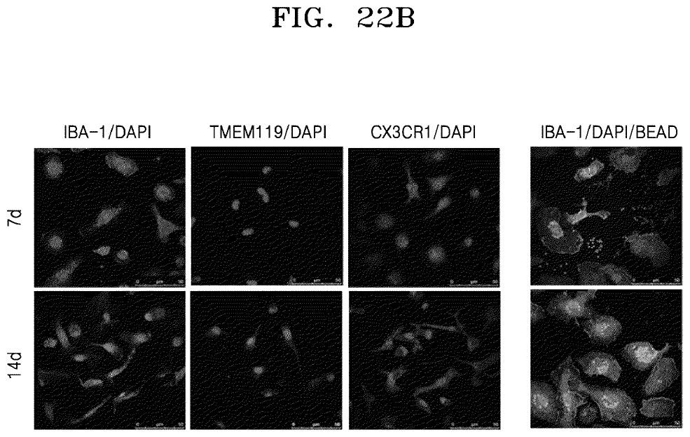

When cultured for 21 days, the microglia changed to a ramified form (FIG. 22A), and it was confirmed that the microglia (NEC-MG), which are isolated by using the MACS system on days 7 and 14 after thawing, well expressed TMEM119 and CX3CR1 and also showed phagocytic activity of microglia (FIG. 22B).

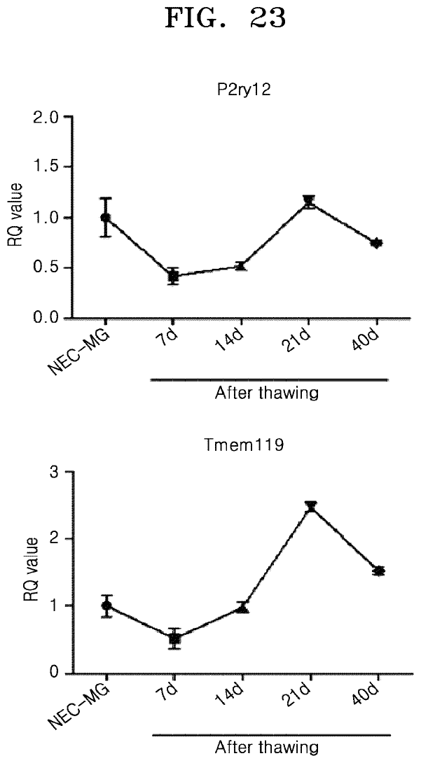

In addition, it was confirmed that P2ry12 and tmem119, which are representative mature microglia genetic factors, were also well maintained when cultured for 40 days (FIG. 23).

1. A method of producing mature microglia, the method comprising:
culturing a mixture including neuroepithelial cells and microglial precursors; and
isolating microglia from the mixture.

2. The method of claim 1, further comprising:
isolating the mixture from a subject's neuroepithelial layer.

3. The method of claim 2, wherein the subject is a fetus isolated from a uterus of a pregnant parent.

4. The method of claim 1, wherein the culturing is subculturing.

5. The method of claim 4, wherein the isolation is performed after 2 to 6 passages.

6. The method of claim 1, further comprising:
freezing and storing the mixture prior to isolating the microglia; and
thawing the mixture.

7. The method of claim 1, wherein the isolating is performed by at least one method selected from the group consisting of a magnetic-activated cell sorting (MACS) system, a fluorescent-activated cell sorting (FACS) system, and a shaking method.

8. The method of claim 1, wherein the microglia express CD11b.

9. The method of claim 1, wherein the microglia express at least one gene selected from the group consisting of IBA-1, Nestin, PU1, TMEM119, P2RY12, CSF1R, MAFB, TREM2, Olfml3, Hexb, TGFbeta, MERTK, C1QA, GPR34, and TGFBR1.

10. The method of claim 1, wherein the microglia are capable of phagocytosis.

11. The method of claim 1, wherein the microglia are in a ramified form.

12. Mature microglial cells, prepared by the method of claim 1.

13. The method of claim 1, wherein the isolating is performed method comprising a magnetic-activated cell sorting (MACS) system, a fluorescent-activated cell sorting (FACS) system, and/or a shaking method.

14. The method of claim 1, wherein the microglia express a gene comprising IBA-1, Nestin, PU1, TMEM119, P2RY12, CSF1R, MAFB, TREM2, Olfml3, Hexb, TGFbeta, MERTK, C1QA, GPR34, and/or TGFBR1.


SEQUENCE LISTING (6 sequences)

SEQ ID NO: 1 - DNA, 20 bp, Artificial Sequence
  Primer GAPDH-F
catggccttccgtgttccta

SEQ ID NO: 2 - DNA, 17 bp, Artificial Sequence
  Primer GAPDH-R
gcggcacgtcagatcca

SEQ ID NO: 3 - DNA, 21 bp, Artificial Sequence
  Primer olfml3-F
ctgctgctcctcttctttttg

SEQ ID NO: 4 - DNA, 20 bp, Artificial Sequence
  Primer olfml3-R
ctactctgatcctggcattg

SEQ ID NO: 5 - DNA, 19 bp, Artificial Sequence
  Primer trem2-F
tgggacctctccaccagtt

SEQ ID NO: 6 - DNA, 18 bp, Artificial Sequence
  Primer trem2-R
gtggtgttgagggcttgg